(19) United States
(12) Patent Application Publication
(10) Pub. No.: US 2005/0182399 A1
(43) Pub. Date: Aug. 18, 2005

(54) Bone fixation device

(76) Inventor: Benjamin Levine, Eden Prairie, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/056,329

(22) Filed: Feb. 11, 2005

Related U.S. Application Data
(60) Provisional application No. 60/544,624, filed on Feb. 13, 2004.

(51) Int. Cl. A61B 17/56

(52) U.S. Cl. 606/60

(57) ABSTRACT

Improved fixation devices for treating, for example, a radius fracture include a first member adapted to engage a distal portion of a radius fracture and a second member operably coupled to the first member adapted to engage a proximal portion of a radius fracture, wherein the first member is shiftable relative to the second member. Due to the adjustability of the first member relative to the second member, the linear distance, between the portion of bone proximal to the fracture and the portion of bone distal to the fracture can be fixed at a desired distance, which can promote suitable healing of the fracture.

### CROSS-REFERENCE TO RELATED APPLICATIONS

The current application claims the benefit of priority from U.S. provisional patent application filed on Feb. 13, 2004, entitled “Distal Radius Fracture Device” having Ser. No. 60/544,624, which is hereby incorporated herein by reference.

### FIELD OF THE INVENTION

The invention relates to treatment and surgical repair of bone fractures. More specifically, the invention relates to surgical repair of long bones such as distal radius fractures that do not involve the articular surfaces of the radius and are minimally comminuted.

### BACKGROUND

Distal radius fractures are the most common fractures presenting to emergency rooms. In 1998, radius fractures were responsible for 2.9 million visits to physicians and accounted for 0.7% of all emergency room visits.

The majority of distal radius fractures occur with the wrist in extension. Commonly, an individual who falls will extend their hands out in from of them to break the fall. Sometimes this causes damage to the bones of the wrist and forearm. The result is that the volar (palm) side of the radius fails in tension while the dorsal (back) side of the radius fails in compression. This results in a simple fracture line of the volar side of the radius and a comminuted fracture pattern on the dorsal side of the radius. The crushed or splintered bone in the comminuted portion of the fractured radius lacks structural integrity so that attempts to realign the fracture portion of the radius to promote healing often fail. The fractured portion of the bone tends to return to its position immediately after the injury occurred due to lack of support. This creates a deformity of the radius that may have several deleterious effects. First, there is visual deformity. Second, there is decreased function due to loss of motion. Third, this deformity increases the stress on certain parts of the articular cartilage, which tends to result in the development of degenerative arthritis in the wrist.

Currently there are several approaches used to repair distal radius fractures. They include closed reduction and casting, the use of percutaneous pins, the application of external fixation and open reduction with internal fixation. While all of these techniques may be appropriate in some circumstances, none is appropriate for every type of fracture or for every patient. Each of the above-mentioned treatment methods must be tailored to the meet the needs of the individual patient. The physician must weigh the risks and benefits of the treatment option to devise a treatment plan that is best suited for each patient.

Closed reduction and casting includes aligning the broken bone ends by external manipulation and applying a cast to the affected limb to immobilize it. The presence of soft tissues between the cast and the fracture allows for some movement of the fractured bone ends relative to each other which is particularly problematic in comminuted fractures where the crushed fracture margins do not meet neatly. Thus, the reduction will often tend toward its post-fracture position. Prolonged immobilization of the wrist joint which can result in stiffness of the wrist after the cast is removed.

Percutaneous pins are passed through the skin and help to bridge the compressed portion of a comminuted fracture. The pins are passed through bone on one side of the break, across the gap and into the bone on the other side of the break. The insertion of percutaneous pins provides some additional support for the break but percutaneous pins also allow some relative movement between the broken bone portions. Percutaneous pins, if buried, must be surgically removed at some later date after healing is complete and if left protruding through the skin must be kept dry and create a risk of infection while present.

External fixators support the fractured bone in proper alignment via external rings and struts that are connected to the bone by rods that pass through the skin and soft tissues generally perpendicular to the bone's long axis. External fixation devices also require that exposed portions of the appliance pass through the skin with attendant concerns of infection. The probes associated with external fixation provide a potential portal for infective agents to pass through the skin. In addition, external fixation may also result in significant joint stiffness after healing.

Internal fixation devices are generally plates that are secured to bone on either side of the fractured area, commonly by screws. The internal fixation device bridge the gap in a comminuted fracture to support the fractured bone ends while healing takes place. Open reduction and internal fixation require a full surgical procedure with the attendant risks and potential for complications. In addition, internal fixation hardware may also require removal at a later date. Removal of the internal fixation device requires a separate surgical procedure, again, with attendant possibility of infection and complication.

It would be beneficial to the orthopedic surgical arts to have another means to stabilizing comminuted fractures of the long bones that is minimally invasive and provides an additional surgical option for those fractures that require more stabilization than a close reduction supplies and yet do not need open reduction with internal fixation.

### SUMMARY OF THE INVENTION

The fixation device of the present invention solves many of the above described problems. The fracture fixation device of the present invention can support many comminuted fractures that need more than closed reduction but do not need open reduction and internal fixation with use of a plate or rod.

The invention disclosed here will be described in the context of reduction (setting) of a comminuted fracture of the distal radius. However, it is to be understood that the present invention can be used to stabilize fractures in other bones at other locations and that the invention is not necessarily limited to used in the distal radius.

An improved fixation device for treating, for example, a radius fracture includes a first member adapted to engage bone distal to a radius fracture and a second member operably coupled to the first member adapted to engage bone proximal to a radius fracture, wherein the first member is axially adjustable relative to the second member. Due to the adjustability of the first member relative to the second member the linear distance between the bone proximal to the comminuted fracture and the bone distal to the fracture can be adjusted to a desired distance, which can promote suitable healing of the fracture and prevent deformity. For example, controlling the linear distance between the distal portion and the proximal portion of the fracture can promote desired healing of fractures having crushed or splintered bone fragments, since the bone ends remain fixed relative to each other throughout the healing process. In some embodiments, the adjustability of the first member relative to the second member can be provided by a ratcheting and/or screw expander operably connected to the first member and the second member. In other embodiments, the first member can include a plurality of openings that facilitate securing the second member to the first member at desired locations.

The fixation devices of the present invention address the needs of patients who suffer from distal radius fractures and solve many of the problems noted above. The fixation devices of the present disclosure can be relatively small, and thus can be positioned within a patient via a small surgical incision. In addition, the fixation devices can provide support of the dorsal aspect of the distal radius where a comminuted fracture has occurred, and therefore can reduce any tendency for the comminuted area to collapse and prevent limb deformity. Moreover, the fixation devices can function like a jack or spacer to keep the area of the fracture open and the distal portion of the fracture properly aligned during the healing process.

In some embodiments, a portion, or all, of the fixation device can be formed from one or more bioresorbable polymers, which can eliminate the need for a second surgery to remove the implanted device.

The fixation devices also provide an additional surgical option that bridges the gap for those fractures that fall between fractures that need nothing more than a close reduction and those fractures that need open reduction with internal fixation in order to achieve the best results for an individual patient

In one embodiment, the invention is a fixation device having a first member, a second member and an expander operably coupled to the first member and the second member. In these embodiments, the first member and the second member can be clip-like support devises that grasp the distal and proximal bone portions at the fracture site, and the expander can be a ratcheting and/or screw mechanism that facilitates adjusting the distance between the first member and the second member.

The expander can comprise a screw mechanism that can be turned by the operating surgeon in order to force the distal clevis and the proximal clevis apart from one another. In another embodiment, the expander can be a ratchet mechanism that can be rotated within an aperture in one or both of the distal and proximal devises in order to allow the distal and proximal devises to be brought closer to one another to facilitate the insertion of the distal and proximal devises within the fractured area of the distal radius.

In yet another embodiment of the invention, the expander may be fixed to either the distal clevis or the proximal clevis with a screw mechanism and fixed to the other clevis by a ratchet mechanism that then can be used to force the distal and proximal devises apart in order to provide stabilization for the comminuted fracture.

In another embodiment, the invention relates to a fixation device having a first member that extends to the subchondral bone at the end of the medullary canal of the distal radius. In these embodiments, the fixation device can further include a second member that is adapted to engage the proximal portion of radius fracture and can slidably engage the first member such that the second member can move or slide along the major axis of the first member. In these embodiments, the first member can include a plurality of openings positioned along the major axis of the first member, which facilitates coupling the second member to the first member at a desired position using a positioning pin.

In another embodiment, the invention relates to an implantable fixation device including a first member adapted to engage with a distal portion of a fracture, and a second member operably coupled to the first member adapted to engage with a proximal portion of a fracture. In these embodiments, the fixation device can be formed from one or more bioresorbable polymers.

In a further embodiment, the invention relates to a method of treating a comminuted fracture, wherein the radius fracture comprises a distal portion and a proximal portion, the method including the step of adjusting a first member relative to a second member such that a desired distance between the distal portion of the fracture and the proximal portion of the fracture is achieved, wherein the first member is engaged with the distal portion of the fracture and the second member is engaged with proximal portion of the fracture, and wherein the first member is operably coupled to the second member.

Another embodiment includes a proximal clevis and an expander. In this embodiment the expander is extended into the distal medullary canal to make contact with the distal subchondral bone to provide support to the fractured radius.

Another embodiment includes a notched rod and a clamp to adjustably fix the expander to the proximal or distal clevis.

### BRIEF DESCRIPTION OF THE DRAWINGS

FIG. 1 is a side sectional view of an embodiment of the present invention in situ in a fractured distal radius;

FIG. 2 is a perspective view of proximal and distal devises in accordance with the present invention;

FIG. 3 is a top plan view of the embodiment of FIG. 1 in situ in a distal radius;

FIG. 4 is a side sectional view of a second embodiment of the present invention in situ in a fractured distal radius;

FIG. 5 is a top plan view of the embodiment of FIG. 4;

FIG. 6 is a top plan view of a third embodiment of the present invention;

FIG. 7 is a side sectional view of the third embodiment in situ in a fractured distal radius;

FIG. 8 is a plan view of a notched rod and a clamp in accordance with the third embodiment of the present invention;

FIG. 9 is a perspective view of another embodiment the present invention having a first member, a second member and a positioning pin;

FIG. 10 is a perspective view of the embodiment of FIG. 9 depicting the second member engaged with first member;

FIG. 11 is a back perspective view of the second member of the embodiment of FIG. 9;

FIG. 12 is a front perspective view of the second member of FIG. 11;

FIG. 13a is a side view of the embodiment of FIG. 9 depicting the second member engaged with a proximal portion of the fracture and the first member inserted into second member;

FIG. 13b is a side view of the embodiment of 13a where the second member has been extended to engage the distal portion of the fracture;

FIG. 14 is a perspective view of an embodiment of a fixation device having a first member with a mushroom shaped end portion.

### DETAILED DESCRIPTION OF THE INVENTION

The invention is described herein in the context of devices and methods for repairing comminuted fractures of the distal radius. This should not be considered limiting. The invention can be used to reduce fractures of other long bones such as, for example, the ulna, humerus, tibia, fibula and the femur.

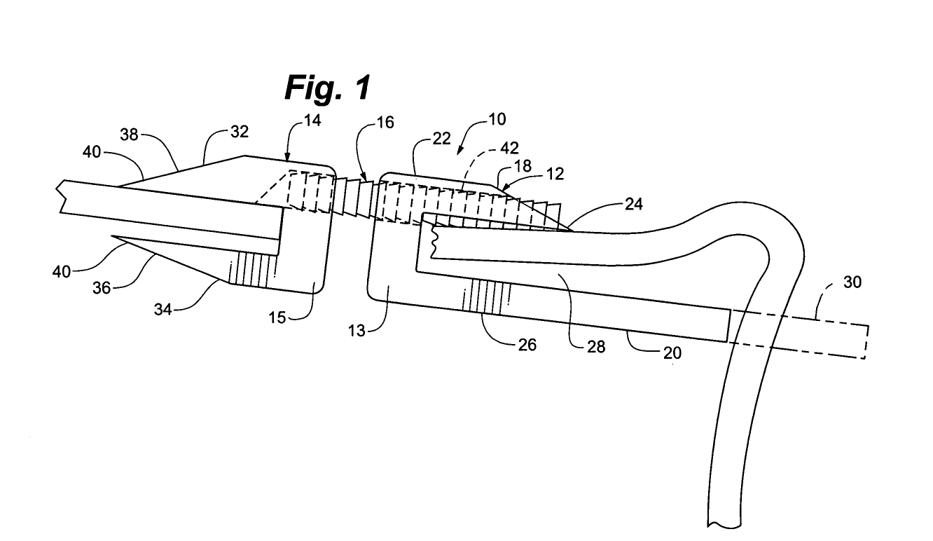

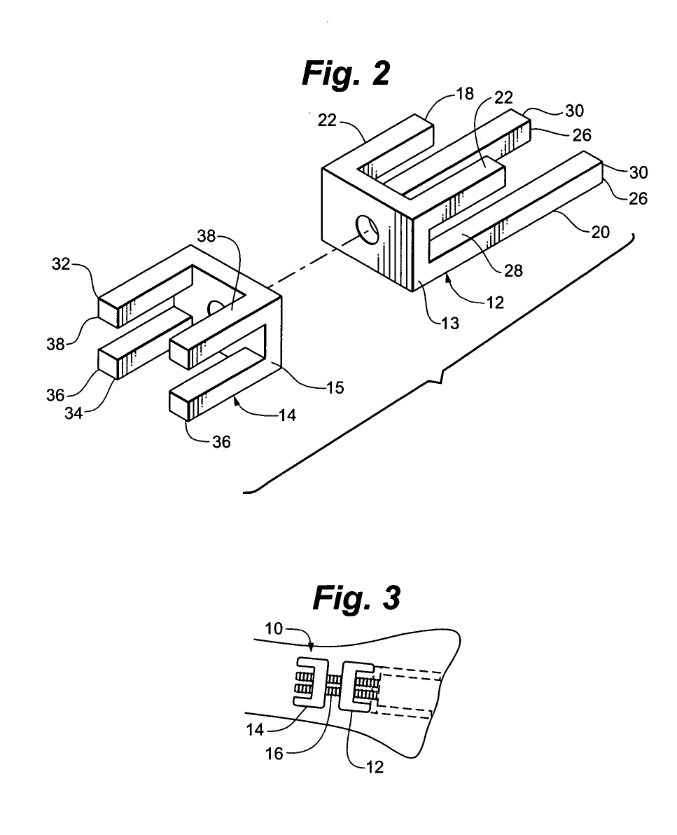

Referring to FIGS. 1 and 2, in one embodiment, fixation device 10 generally includes first member 12, second member 14 and expander 16. As depicted in FIGS. 1 and 2, first member 12 may be a clip-like distal clevis 13 having outer fork 18 and inner fork 20. Outer fork 18 desirably has two legs 22 that terminate in a beveled tip 24. Distal clevis 13 can also includes inner fork legs 26. Outer fork legs 22 are separated from inner fork legs 26 by space 28. Space 28 is sufficient to accommodate the thickness of the cortical bone supported, which, in this example, is the cortical bone of the distal radius. Typically, the cortical bone of the distal radius is about two millimeters thick, thus the space between outer fork legs 22 and inner fork legs 26 is typically about two to about four millimeters. However, the space between the outer fork legs 22 and the inner fork legs 26 can be from about one to about ten millimeters. The size of the space between the inner fork legs and the outer forks legs can be guided by the thickness of the bone that a particular fixation device is designed to engage.

Inner fork legs 26 can have an extended length relative to outer fork legs 22 and may terminate in a blunt end 30. The extended length of inner fork legs 26 is sufficient to extend well beyond the expected distance from a comminuted fracture from the distal end of the medullary canal in a distal radius or other bone to be repaired. This allows for inner fork legs 26 to be trimmed to an appropriate length to abut the distal end of the medullary canal in a fractured distal radius or other bone.

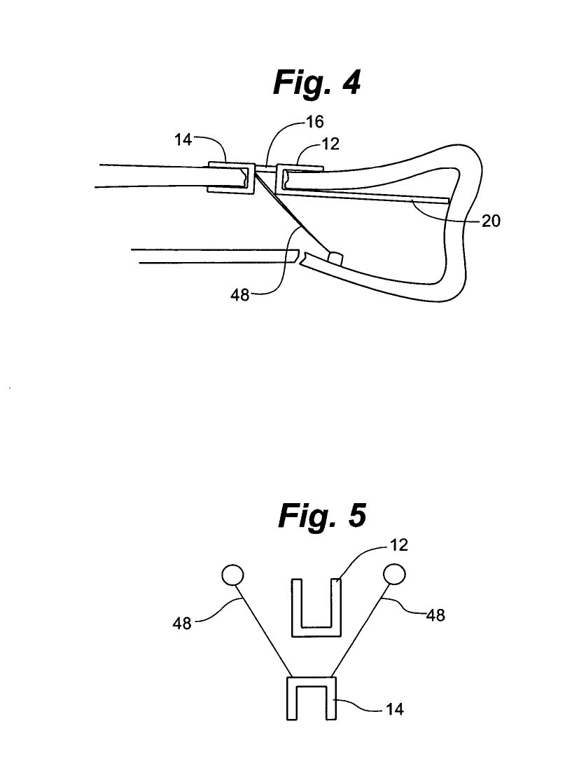

As depicted in FIGS. 1 and 2, second member 14 may include a clip-like proximal clevis 15 having outer fork 32 and inner fork 34. In some embodiments, outer fork 32 and inner fork 34 are desirably substantially mirror images of one another. Inner fork 34 may have two inner legs 36 of substantially similar length and construction. Outer fork 32 desirably includes two outer legs 38 which are substantially of similar construction. Both inner legs 36 and outer legs 38 desirably end in beveled tip 40. Referring to FIGS. 4 and 5, in some embodiments, the proximal clevis can further include struts 48. In one embodiment, struts 48 extend distally, downwardly and outwardly from the proximal clevis so as to reach into and across the medullary cavity when fixation device 10 is in situ. Thus, in this embodiment, fixation device 10 provides additional stabilization of the distal radius by supporting the internal volar side of the distal radius via struts 48.

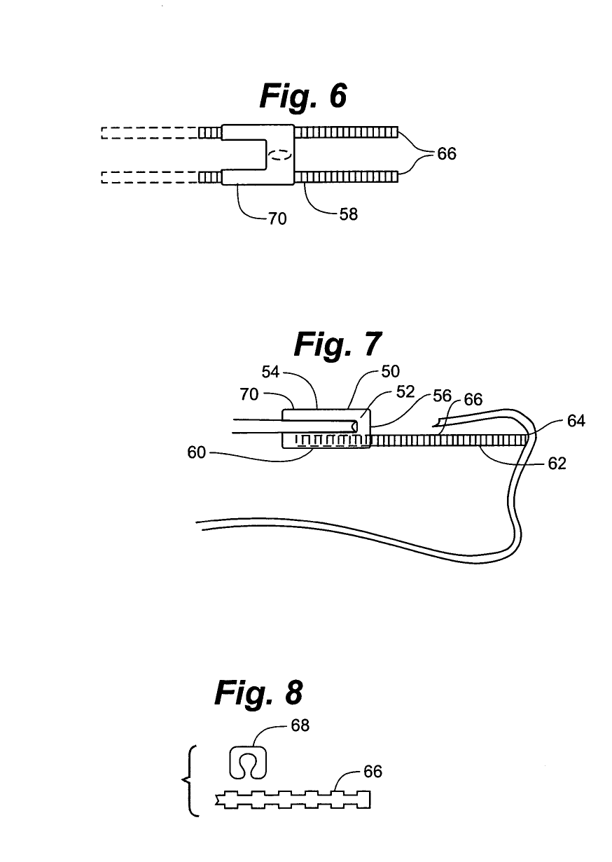

Referring to FIGS. 6, 7 and 8, in some embodiments, fixation device 10 can include a proximal clevis 50 having an L-shaped structure 52. Proximal clevis 50 generally includes outer fork 54 and flange 56. Flange 56 is oriented at a generally right angle to outer fork 54. In this embodiment expander 58 interfaces directly with flange 56 and extends within the medullary canal proximal to the fracture to engage the distal radius cortex between the proximal portion 60 of the expander 58 so that the bone cortex is held between the proximal portion 60 and outer fork 54. As depicted in FIG. 7, distal portion 62 of the expander 58 extends distally through the medullary canal until the distal end 64 of the expander 58 comes into contact with subchondral bone at the end of the medullary canal.

As described above, fixation device 10 can include an expander 16 interposed between the first member 12 and the second member 14, to facilitate moving first member 12 and second member 14 apart, to support the two halves of the comminuted fractured portion of the distal radius and prevent the fractured portions of the bone from returning to their post fracture alignment.

In one embodiment, expander 16 may include a ratchet 42. In this example, expander 16 will be referred to as being secured to second member 14 and to be slidably fixable relative to first member 12. However it is to be understood that expander 16 may be fixed to either first member 12 or second member 14 and may be slidingly engagable to either first member 12 or second member 14.

In other embodiments, expander 16 may be threaded so that one end has a right hand thread and the other end has a left end thread. Thus, the expander may be threadably engaged to first member 12 at a first end 44 and to second member 14 at a second end 46. In these embodiments, expander 16 may be turned so as to force first member 12 away from second member 14 in an operation similar to the operation of a turnbuckle.

Referring to FIG. 8, expander 58 may take the form of a notched rod 66. Notched rod 66 may be secured relative to proximal clevis 50 by clamp 68. In addition, in another embodiment, proximal clevis 50 may engage with two notched rods 66. Thus, in situ, the bone cortex is gripped between two prongs 70 of outer fork 54 and two notched rods 66. Additional expander mechanisms are contemplated and are within the scope of the present disclosure.

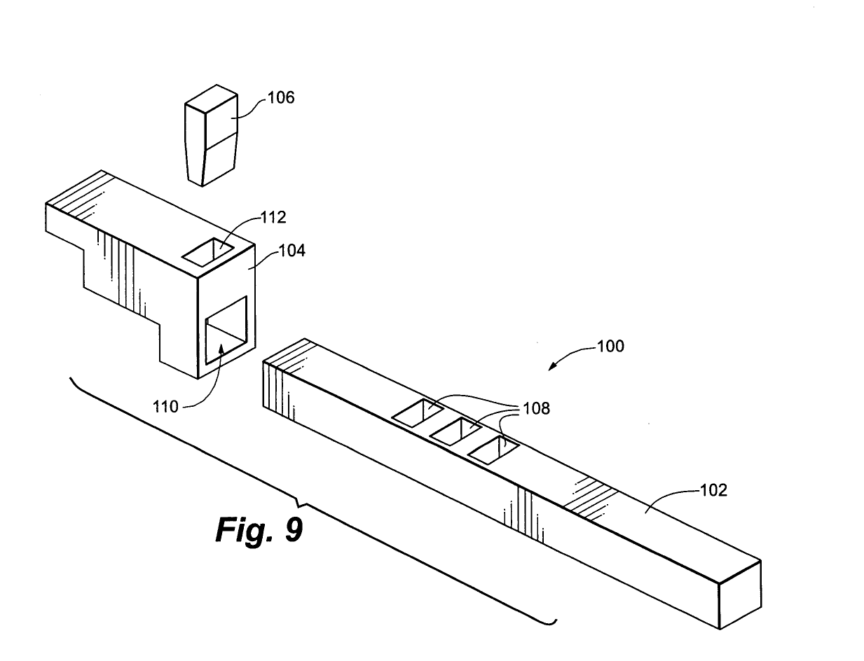

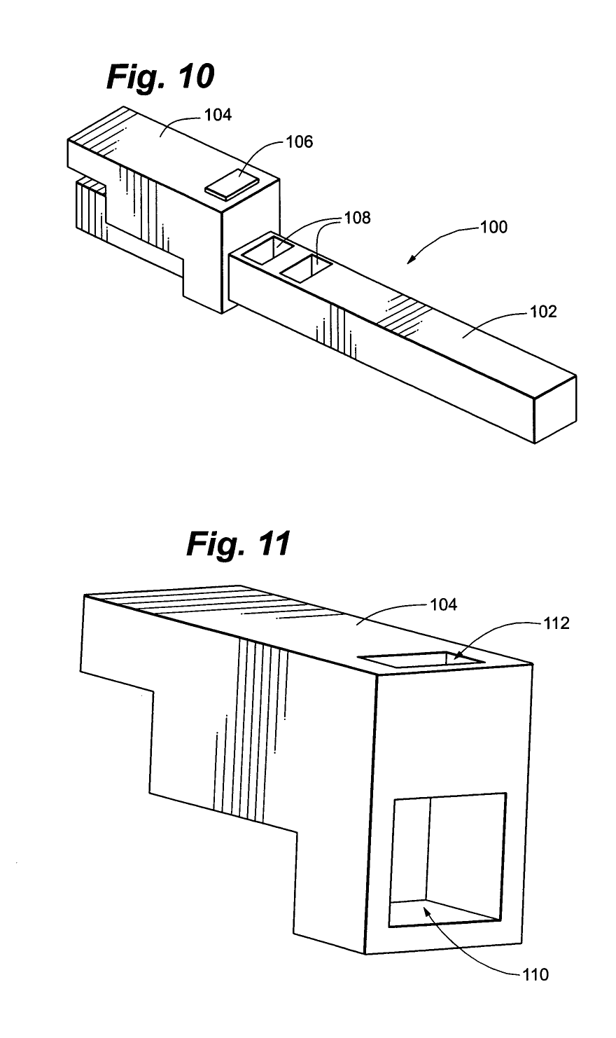

Referring to FIGS. 9 and 10, another embodiment of a fixation device 100 is depicted including first member 102, second member 104 and positioning pin 106. First member 102 can be adapted to slideably engage second member 104, which facilitates coupling the first member to the second member at desired locations. As depicted in FIGS. 9 and 10, first member 102 can have an elongate major axis relative to a minor axis, and can include a plurality of openings 108 positioned along the major axis.

Generally, second member 104 can include first opening 110, which is adapted to receive first member 102 and facilitates slidably engaging first member 102 and second member 104. In some embodiments, first opening 110 can have a rectangular cross-section, while in other embodiments first opening 110 can have an circular cross-section, an oval cross-section or other cross sectional shapes.

Second member 104 can also include second opening 112, which is adapted to receive positioning pin 106. As depicted in FIG. 10, positioning pin 106 can be inserted into second opening 112 and extend into one of the plurality of openings in first member 102 to coupled second member 104 to first member 102.

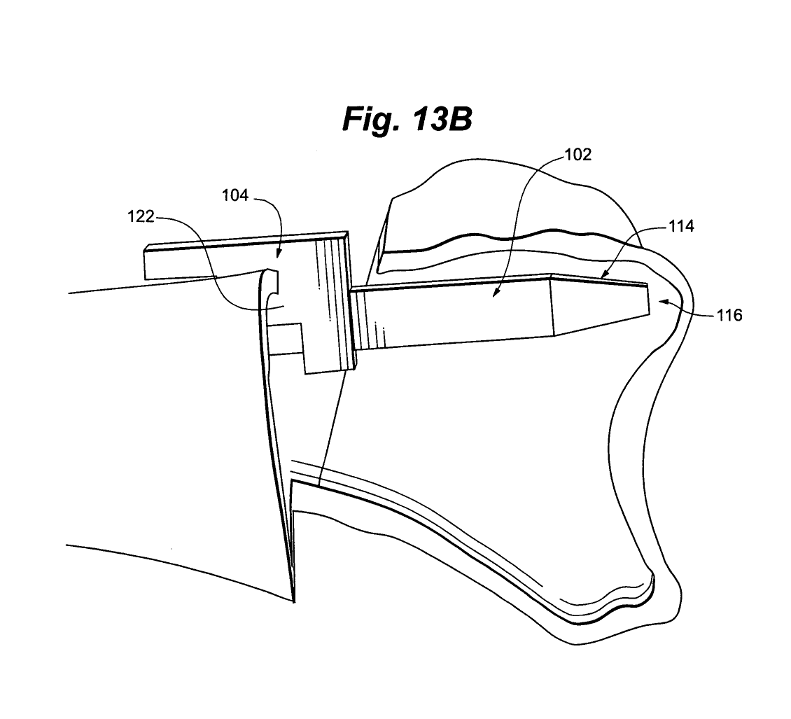

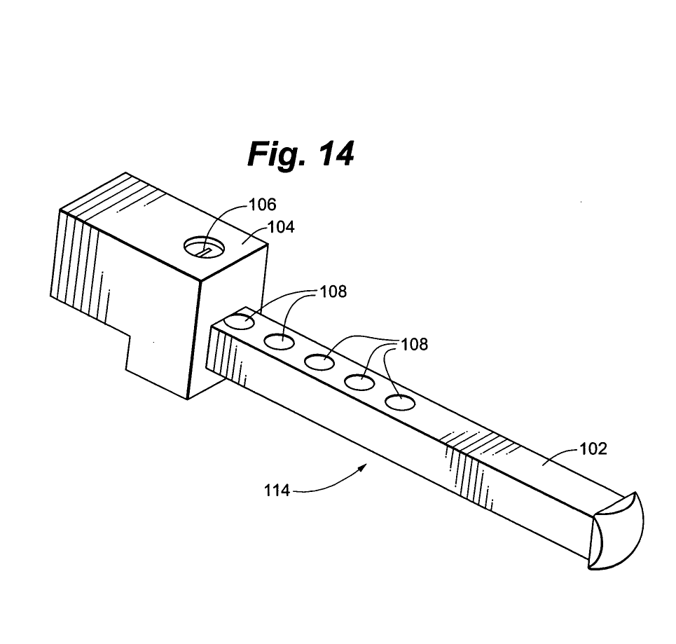

As depicted in FIGS. 9-10, first member 102 may include a rod having an elongated major axis relative to a minor axis. In some embodiments, first member 102 can have a rectangular cross-section, a circular cross-section, an oval cross-section or other cross sectional shape. The size and cross-sectional shape of first member 102 can be guided by the corresponding size and shape of first opening 110 on second member 104. As depicted in FIGS. 13a and 13b, in some embodiments, end portion 114 of first member 102 can be tapered to facilitate engagement with the medullary canal 116 of the distal radius. As depicted in FIG. 14, end portion 114 can present a mushroom shape, a dome shape or the like to facilitate engagement with the medullary canal 116 of the distal radius.

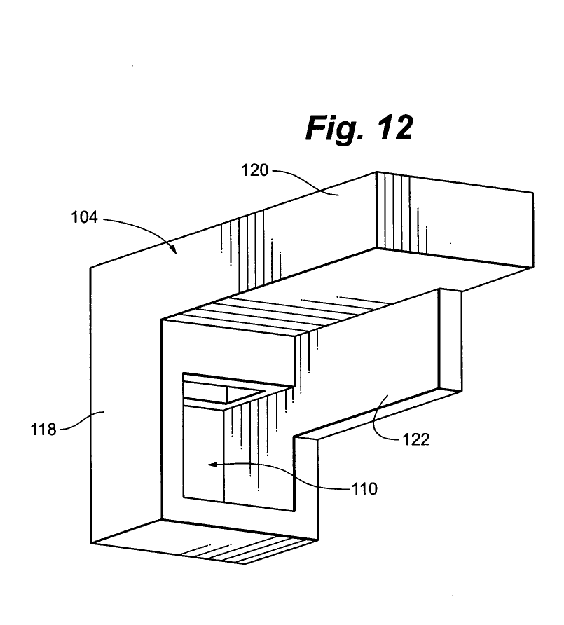

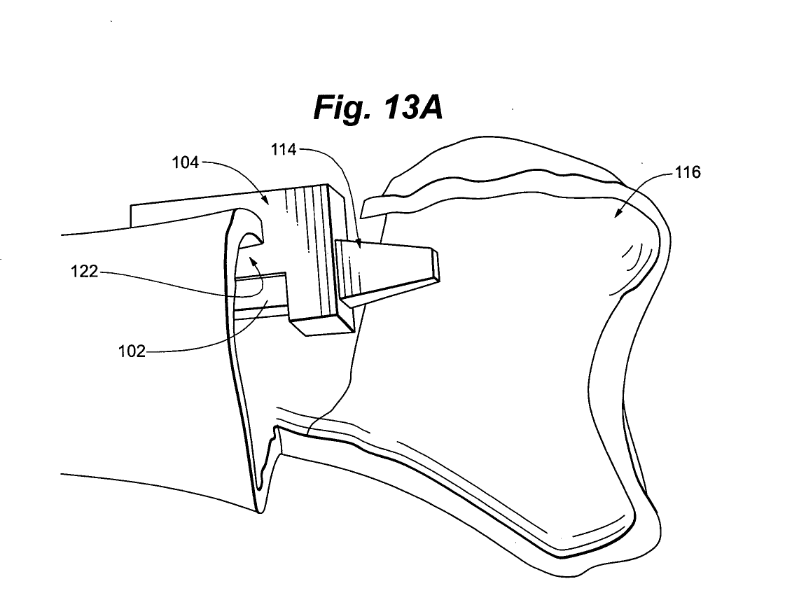

As described above, second member 104 is designed to engage the proximal portion of a radius fracture. Referring to FIGS. 11 and 12, in some embodiments, second member 104 can be a L-shaped bracket having a first leg 118 connected to a second leg 120. First leg 118 includes first opening 110 adapted to receive first member 102, while second leg 120 includes second opening 112 adapted to receive positioning pin 106. As depicted in FIG. 12, a support plate 118 can be positioned adjacent to opening 110, which can help support the L-shaped bracket when the bracket is under a load. Additionally, support plate 122 can be positioned into a notch cut into the proximal portion of the radius, or other bones, to prevent second member 104 from moving out of a desired location during healing of the fracture. FIG. 13 depicts support plate 122 positioned within a notch in the proximal portion of a radius fracture to prevent second member 104 from shifting during healing of the fracture. In some embodiments, support plate 122 can have a length that is coextensive with a length of second leg 120, while in other embodiments, as depicted in FIG. 12, second leg 120 can extend beyond support plate 122.

In operation, fracture stabilizer 10 is applied to a comminuted fracture, for example, of the distal radius. Once the surgeon has surgically accessed the fractured area of the radius, second member 14 is inserted so that the cortex of the proximal portion of the fractured radius is inserted between outer fork 32 and inner fork 34. Similarly, first member 12 is inserted into the portion of the fractured radius distal to the break, so that outer fork 18 and inner fork 20 surround the cortex of the distal fractured radius. First member 12 and second member 14 are then aligned so that expander 16 may be interposed between the two. Expander 16 is then adjusted to force first member 12 away from second member 14 until the fractured radius is aligned as desired to allow for proper healing of the fractured bone.

To employ fixation device 100, a small incision is made on, for example, the dorsal wrist adjacent the fracture. A small notch, or cut, is then made in the proximal portion of the bone where second member 104 is to be positioned.

First member 102 can then be inserted into first opening 110 of second member to make fixation device 100 more compact. Fixation device 100 can then be placed in the gap of a comminuted fracture such that support plate 122 of second member 104 slips into the notch into the proximal portion of the bone. Once second member 104 is engaged with the proximal portion of the fracture, first member 102 can be extended and extended through the marrow to the distal end of the intramedullary canal to abut the subchondral bone.

Positioning pin 106 is then inserted into second opening 112 and extended into an opening 108 located on first member to secure second member 104 to first member 102.

The fixation devices of the present disclosure can be formed from any biocompatible material suitable for orthopedic implants including metals, metal alloys, polymers, bioresorbable polymers and combinations thereof. Suitable metals include, for example, consisting of stainless steel, titanium, alloys of iron, cobalt, nickel, tantalum, zirconium, silver, gold, alloys of copper, platinum, palladium and alloys and combinations thereof.

Suitable polymers may include, for example, polyesters, polyanhydrides, polycarbonates, polyurethanes, polyphosphazenes, polyamino acids, polycyanocrylates, polyphosphazenes, and blends and combinations thereof.

The fixation devices may be constructed from one or more bioresorbable polymers, which can eliminate the need for removal. For the purposes of this application the term bioresorbable is considered to include materials that are incorporated into the living tissue as well as materials that are broken down and excreted by the body. Bioresorbable polymers are well known the medical arts. Suitable bioresorbable polymers include, for example, poly(glycolic acid) (PGA), poly(d,1-lactic-co-glycolic acid), poly(caprolactone), poly(propylene fumarate), poly[1,6-bis(carboxyphenoxy) hexane], tyrosine-derived polycarbonate, polyurethane based on LDI and poly (glycolide-co-γ-caprolactone), ethylglycinate polyphosphazene, poly(dioxanone) (PDS), poly(hydroxybutyrate) (PHB), poly(hydroxyvalerate) (PHV), poly(1-lactic acid) (PLLA), poly(d,1-lactic acid) (PDLA) and combinations thereof.

The embodiments above are intended to be illustrative and not limiting. Additional embodiments may be found within the claims. Although the present invention has been described with reference to particular embodiments, workers skilled in the art will recognize that changes may be made in form and detail without departing from the spirit and scope of the invention.

1. A fixation device to assist in the reduction of comminuted fractures, comprising:
a first member engagable to a distal portion of bone adjacent a bone fracture; and
a second member operably coupled to the first member and engagable to a proximal portion of bone adjacent the bone fracture,
wherein the first member is shiftable between a first position and a second position relative to the second member and fixable at the second position such that a linear distance between the proximal portion of bone and the distal portion of bone can be substantially fixed.

2. The fixation device of claim 1, wherein the first member comprises a distal clevis clip having an outer fork and an inner fork, wherein the outer fork and the inner fork each comprise two legs.

3. The fixation device of claim 2, wherein the legs of the outer fork terminate in a beveled tip.

4. The fixation device of claim 2, wherein the outer fork legs and the inner fork legs are separated by a space that is from about 1 millimeter to about 6 millimeters.

5. The fixation device of claim 2, wherein the inner fork legs have an extended length relative to the length of the outer fork legs.

6. The fixation device of claim 2, wherein the inner fork legs terminate in a blunt end.

7. The fixation device of claim 1, wherein the second member comprises a proximal clevis clip having an outer fork and an inner fork, wherein the outer fork and the inner fork each comprise two legs.

8. The fixation device of claim 7, wherein the legs of the outer fork and the legs of the inner fork end in a beveled tip.

9. The fixation device of claim 7, wherein the legs of the outer fork have substantially the same length as the legs of the inner fork.

10. The fixation device of claim 7, wherein the proximal clevis clip further comprises one or more struts extending distally, downwardly and outwardly from the proximal clevis.

11. The fixation device of claim 1, wherein the second member comprises an L-shaped proximal clevis.

12. The fixation device of claim 1, further comprising an expander operably coupled to the first member and the second member to facilitate shifting of the first member relative to the second member.

13. The fixation device of claim 12 wherein the expander is selected from the group consisting of a screw mechanism, a ratchet mechanism or a combination thereof.

14. The fixation device of claim 1 wherein the first member and the second member are composed of a material selected from the group consisting of metals, metal alloys, polymers, bioresorbable polymers and combinations thereof.

15. The fixation device of claim 1 wherein the first member and the second member are composed of a polymer selected from the group consisting of poly(glycolic acid) (PGA), poly(d,1-lactic-co-glycolic acid), poly(caprolactone), poly(propylene fumarate), poly[1,6-bis(carboxyphenoxy) hexane], tyrosine-derived polycarbonate, polyurethane based on LDI and poly (glycolide-co-γ-caprolactone), ethylglycinate polyphosphazene, poly(dioxanone) (PDS), poly(hydroxybutyrate) (PHB), poly(hydroxyvalerate) (PHV), poly(1-lactic acid) (PLLA), poly(d,1-lactic acid) (PDLA) and combinations thereof.

16. The fixation device of claim 1 wherein the first member and the second member are composed of a metal selected from the group consisting of stainless steel, titanium, alloys of iron, cobalt, nickel, tantalum, zirconium, silver, gold, alloys of copper, platinum, palladium and alloys and combinations thereof.

17. A method of reducing a comminuted fracture, the method comprising the steps of:
surgically accessing the fracture;
engaging a first member to a distal portion of bone adjacent the fracture; and
engaging a second member to a proximal portion of bone adjacent the fracture;
operably coupling the second member to the first member,
shifting the first member between a first position and a second position relative to the second member; and
fixing the first member at the second position such that a linear distance between the proximal portion of bone and the distal portion of bone is substantially fixed.

18. The method of claim 17, in which the second member is engaged to subchondral bone at the end of the intramedullary canal.

19. The method of claim 17, in which the second member is engaged to cortical bone along sides of the intramedullary canal.
